(19) United States
(12) Patent Application Publication
(10) Pub. No.: US 2008/0058805 A1
(43) Pub. Date: Mar. 6, 2008

(54) Spinal fusion implant

(75) Inventor: J. Michael Stuart, Rio Rancho, NM (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: MicroDexterity Systems, Inc., Albuquerque, NM (US)

(21) Appl. No.: 11/511,017

(22) Filed: Aug. 28, 2006

(51) Int. Cl. A61F 2/30

(52) U.S. Cl. 606/61

(57) ABSTRACT

A spinal fusion implant system and a method of using the spinal fusion implant system are provided. The spinal fusion implant system includes a plurality of links with each link having a pair of opposing end portions. The spinal fusion implant system also includes a plurality of pedicle screws. Each pedicle screw has a head and a depending shank for fixing the pedicle screw to a bone. The head has a slotted recess configured to receive one end portion of at least two different links or two slotted recesses each of which is configured to receive an end portion of one of the links. Each recess has an associated set screw for clamping the end portions of the respective links in the recess.

### FIELD OF THE INVENTION

The present invention relates to implants that can be used to perform spinal fusions and other procedures where vertebrae are linked together by a rod that is routed through the heads of pedicle screws.

### BACKGROUND OF THE INVENTION

Spinal fusion is a well-developed open surgical procedure in which vertebrae are linked together by a rod that is routed through the heads of pedicle screws that are attached to each vertebra. In particular, the pedicle screws have a slot in the head to accommodate a rod that passes from one screw to the other. The rod is secured to each pedicle screw with a second set screw component. This set screw binds to the rod and holds the vertebrae in a specific orientation and alignment.

Recently, devices have been developed that can perform these spinal fusion procedures with less invasion of the tissue. Such devices only require ports to place the screws and a “needle puncture” to insert a tie rod through the heads of the pedicle screws. Because the tie rod has to have an arc shape, this technique generally has been only used for securing two and three vertebrae in a procedure. It becomes quite difficult to extend this technique beyond the range of two to three vertebrae. If the arc of the tie rod is made larger, then the insertion point becomes increasingly further away from the screw heads and collateral tissue damage occurs.

### BRIEF SUMMARY OF THE INVENTION

According to the present invention, the long continuous rod used in conventional spinal fusion procedures is replaced with short segments that can be inserted through the same types of access ports used to install the pedicle screws. Each rod segment has the ability to reach from one pedicle screw to the next. The pedicle screw is used to clamp on to the rod segments. The rod segments can take a variety of shapes from simple cylindrical short rods to sophisticated coined and forged shapes with special heads and features to lock one rod segment to the next.

### BRIEF DESCRIPTION OF THE SEVERAL VIEWS OF THE DRAWINGS

FIG. 1 is an exploded perspective view of an exemplary spinal fusion implant system according to the invention.

FIG. 2 is top perspective view of an exemplary fixation using the spinal fusion implant system of FIG. 1.

FIG. 3 is an enlarged partial side view of a conventional pedicle screw and the corresponding mating rod segments.

FIG. 4 is a plan view of an alternative embodiment of a rod segment for a spinal fusion implant system according to the present invention.

FIG. 5 is a side view of the rod segment of FIG. 4.

FIG. 6 is an exploded perspective view of a spinal fusion implant system according to the present invention using a pedicle screw with a single set screw and the rod segment of FIGS. 4 and 5.

FIG. 7 is a plan view of another embodiment of a rod segment for a spinal fusion implant system according to the present invention.

FIG. 8 is a side view of the rod segment of FIG. 7.

FIG. 9 is a side view of a spinal fusion implant system using the rod segment of FIGS. 7 and 8.

FIG. 10 is a plan view of another embodiment of a rod segment for a spinal fusion implant system according to the present invention.

FIG. 11 is an exploded perspective view of the rod segment of FIG. 10.

FIG. 12 is a side view of a pedicle screw for use with the rod segment of FIG. 10.

FIG. 13 is a further side-view of an alternative embodiment of the pedicle screw of FIG. 12.

FIG. 14 is a side view of a spinal fusion implant system using the rod segment of FIG. 10.

FIG. 15 is partial perspective view of an alternative embodiment of a rod segment for a spinal fusion system according to the present invention.

FIG. 16 is a side view of a spinal fusion implant system using the rod segment of FIG. 15.

FIG. 17 is a perspective view of another embodiment of a rod segment for a spinal fusion implant system according to the present invention.

FIG. 18 is a perspective view of a variation of the rod segment of FIG. 17.

FIG. 19 is a side view of a spinal fusion implant system using the rod segment of FIG. 18.

FIG. 20 is a partial side view of the head of a pedicle screw for use in a spinal fusion implant system according to the present invention.

FIG. 21 is a partial side view of the head of an alternative embodiment of a pedicle screw for use in a spinal fusion implant system according to the present invention.

### DETAILED DESCRIPTION OF THE INVENTION

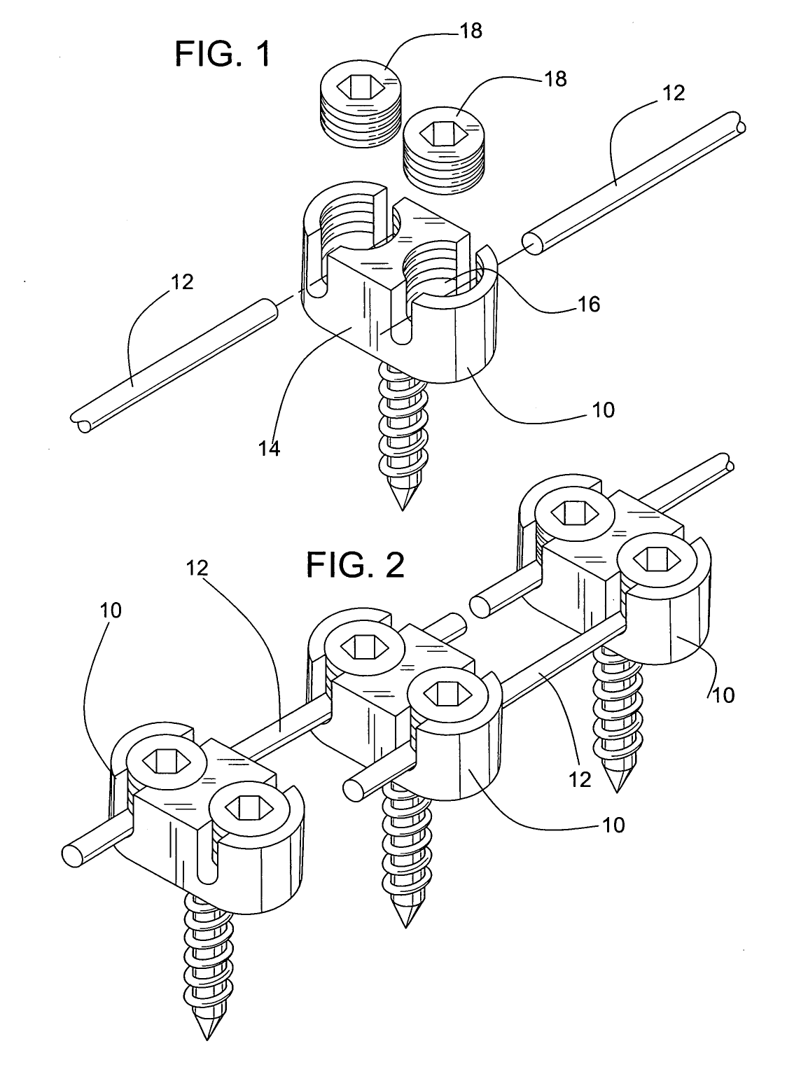

Referring to FIG. 1 of the drawings there is shown an exemplary embodiment of a spinal fusion system according to the present invention. The system of FIG. 1 includes one or more pedicle screws 10 and one or more links or rod segments 12. Each pedicle screw includes a head portion and a depending shank portion that is used to secure the pedicle screw to bone. The rod segments 12 can take any desired shape or configuration sufficient to reach from one pedicle screw to the next, such as, for example, cylindrical or flat rods, bars or wires. Advantageously, the rod segment 12 of FIG. 1 has a very simple, substantially straight configuration.

Each of the pedicle screws 10 used with the rod has a generally oval, figure-eight configured head 14 that defines two separate slotted recesses 16 each of which can receive a rod segment 12. To this end, each of the slotted recesses 16 has opposing notches in the sidewall of the recess through which a rod segment can be inserted. Additionally, each of the slotted recesses 16 in the head 14 of the pedicle screw 10 is threaded so as to be able to receive a corresponding set screw 18.

During a surgical procedure, access holes are first created for the setting of the pedicle screws 10 into the vertebrae. The access holes or ports can be relatively small in size, e.g. as small as 1 cm, so that the procedure is minimally invasive. When the screws are set, the heads 14 of the pedicle screws 10 are aligned such that one of the slotted recesses 16 in the head is located medially to the spine and the other recess 16 is oriented laterally to the spine. Once pedicle screws 10 have been inserted in two adjacent vertebrae, a rod segment 12 can then be inserted through the access hole for one of the pedicle screws 10 and placed in either the left or right slotted recesses 16 of the screws 10. The rod segment 12 is then secured in the slotted recesses 16 via the set screws 18.

As shown in FIG. 2, if a third vertebra is to be involved in the procedure and secured to the previous two, then a third pedicle screw 10 would be set into the third vertebra. The rod segment 12 would be inserted as before through the access port used to place the pedicle screw 10 and routed through the unused slotted recess 16 from the second vertebra pedicle screw and the corresponding slotted recess on the third vertebra pedicle screw. Set screws 18 would be used to secure the rods in the recesses of the pedicle screws.

This procedure can be continued as necessary to fix more vertebrae together. The rod segments 12 alternate from side to side of the pedicle screw heads 14 as the fixation is built from one level to the next. The two vertebrae on the end of the fixation will only have one rod segment 12 each and the associated pedicle screws 10 will have an unused slotted recess 16. Each intermediate level in the fixation will have two rod segments 12 in each pedicle screw 10. One of these intermediate rod segments 12 will be on the left side and be secured to the left side of the next level and the other rod segment will be on the right side and be secured to the right side of the opposite next level. Alternatively, the rod segments 12 could extend diagonally with the rod segments extending, for example, from the left side of one pedicle screw 10 to the right side of the next pedicle screw 10 in the chain. As opposed to the multiple rod segments 12, the pedicle screws 10 of FIGS. 1 and 2 could also be, used with parallel, continuous multilevel wires such as are used in conventional open surgical procedures.

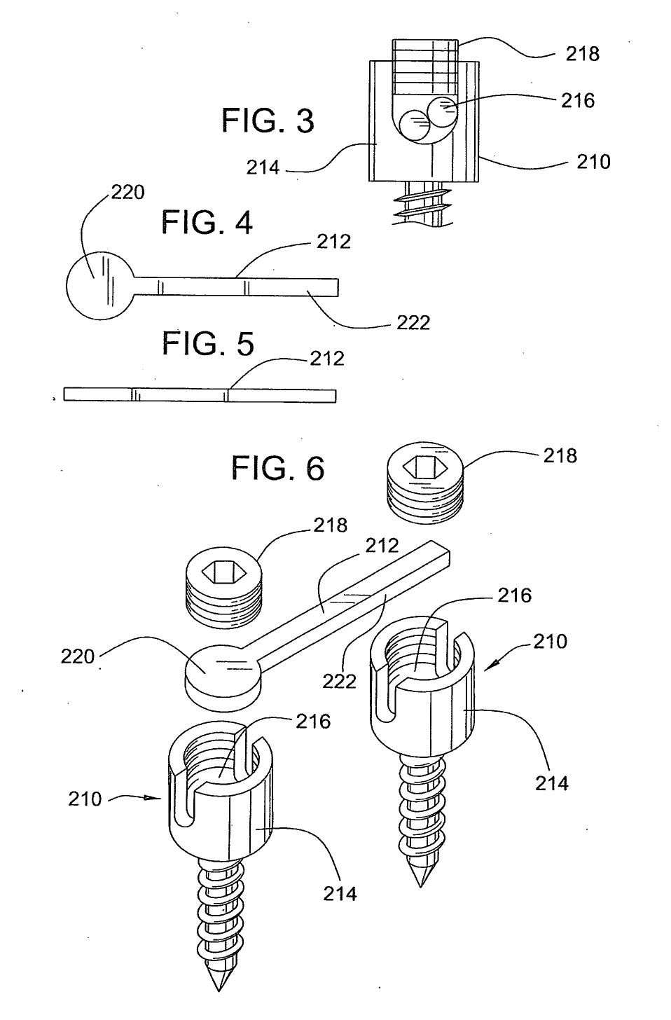

Conventional pedicle screws typically have a more compact head than the pedicle screws used in the embodiment of FIGS. 1 and 2. In particular, conventional pedicle screws 210 as shown in FIG. 3 generally have only a single set screw 218 to fix the rod to a slotted recess 216 in the head of the pedicle screw. Moreover, conventional pedicle screws have a much more symmetrical and coaxial structure. These conventional set screws generally work fine for most current open spinal fusion surgical procedures.

While the multiple rod segments of the embodiment of FIGS. 1 and 2 can be used with conventional pedicle screws 210 such as shown in FIG. 3, such use can place a great deal of stress on the pedicle screw head that could eventually lead to the failure of the fixation at one or more levels. In particular, when clamping more than one rod segment in the slotted head of the pedicle screw 210, the wires will tend to wedge each other apart if the wires are less than the width of the slot in the pedicle screw head. Rods that are a close fit for the pedicle screw slot will have one round rod segment stacked on top of another leading to high contact forces between wires, high stress and the potential to lose fixation when the rod segments deform because of the high stress. Once the rod segments start to compress and loosen, then the situation will get progressively worse. In addition, a pedicle screw that is sized large enough to accommodate two rod segments will have a taller head than a pedicle screw that only has to accommodate one rod.

To help ensure that the rod segments will stay fixed to the pedicle screw 210 and one another with a nominal amount of pressure from the set screw 218 in the pedicle screw head 216, the rod segments can have special shapes, material properties, and/or surface textures. For example, one or both ends of the rod segments could have a D-shape configuration, knurling, flattened portions, grooves or any combination thereof. One exemplary special rod segment design is illustrated in FIGS. 4 and 5. The rod segment 212 of FIGS. 4 and 5 has a banjo shape including an enlarged head portion 220 and a smaller elongated neck portion 222. The banjo shape can be produced by coining a metal blank. The metal blank can be produced from wire or sheet metal. The enlarged head portion 220 of the rod segment 212 is sized and configured to fit into the corresponding slotted recess 216 in the head of the pedicle screw 210 (see, e.g., FIG. 6). As with the rod segments 12 of the embodiment of FIGS. 1-2, the banjo shaped rod segment 212 can be inserted through the same small access hole that would be used for the placement of the pedicle screw 210. The enlarged head portion 220 of the rod segment 212 would then be clamped within the head 214 of the pedicle screw 210 as shown in FIG. 6. The neck portion 222 of the banjo shaped rod segment 212 would extend toward another vertebra and would be clamped there in the slotted recess 216 of a second pedicle screw 210 in order to fix the two adjacent vertebrae together.

The banjo shaped rod segments 212 have a generally flat shape that allows the rod segments to be stacked on top of one another in the slotted recesses 216 of the pedicle screws 210 (see, e.g., FIG. 9). This allows a further rod segment 212 to be placed on top of the previous rod segment 212 in order to fix more than two vertebrae together. In particular, the enlarged head portion 220 of the further rod segment can be placed over the neck portion 222 of the previous rod segment. The two rod segments 212 are then compressed together in the slotted recess 216 in the pedicle screw 210 via the set screw 218. The enlarged head portion 220 of the second rod segment 212 would press on the first rod segment and the set screw 218 would lock them together. Since the walls of the slotted recess 216 in the pedicle screw 210 taper inwardly as they extend towards the bottom of the recess (see, e.g., FIG. 3), the set screw 218 would force the neck portion 222 of the first rod segment 212 into a more closely confined space in the recess. The locking of the two rod segments 212 can be enhanced by material properties and surface texture and features. For example, hardened teeth in the surface of the enlarged head portion 220 of the second rod segment 212 would enhance the grip on the neck portion 222 of the first rod segment.

The neck portion 222 of the second rod segment 212 can be extended toward a third pedicle screw 210. As described previously, the procedure may be completed at the third pedicle screw 210 by simply inserting and tightening the set screw 218. Alternatively, the procedure could be continued to additional levels by adding more pedicle screws 210 and banjo shaped rod segments 212 in the order and orientation described above.

The banjo shaped rod segments 212 can be used to fix any number of vertebrae together. Advantageously, the pedicle screws 210 can have a configuration very similar to conventional screws. However, unique features can be provided in the slotted recesses 214 in the pedicle screws 210 to enhance gripping of the banjo shaped rod segments 212, and in particular the neck portion 222 of the rod segments. For example, the slotted recess 214 could be provided with a V-shaped or closely confining square bottom 24 (see FIGS. 20 and 21) with a less restrictive space higher up in the recess to allow for the enlarged head portion 220 of the rod segment 212.

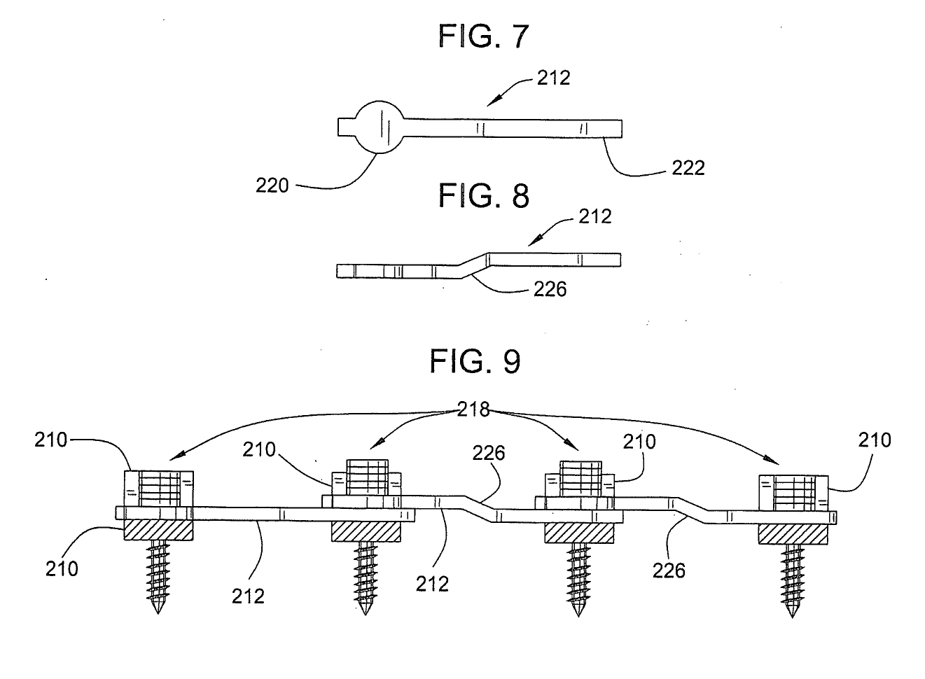

According to a further embodiment of the invention, the banjo-shaped rod segments 212 can have a non-linear configuration. In particular, as shown in FIGS. 7 and 8, an offset 226 can be provided in the rod segment 212. The amount of the offset 226 can correspond to the metal thickness of the neck section 220 of the banjo shaped rod segment 212. As shown in FIG. 9, such an offset 226 will allow straight fixation columns to be built without worrying about the angular misalignment introduced by the slight offset in height from one pedicle screw to the next.

The banjo-shaped rod segments 212 can have a variety of different sizes to accommodate patients of varying size. Until the set screw 218 is tightened, the neck portion 222 of the rod segment can slide longitudinally in the slotted recess 216 in the pedicle screw 210 to allow for adjustment or alignment of the vertebrae. Moreover, the adjacent rod segments 212 can overlap so as to allow for adjustment and anatomy variations from one patient to the next. The overlap of the rod segments 212 also allows the number of different sizes to be reduced since there is an inherent adjustable nature to the overlapping rod segments.

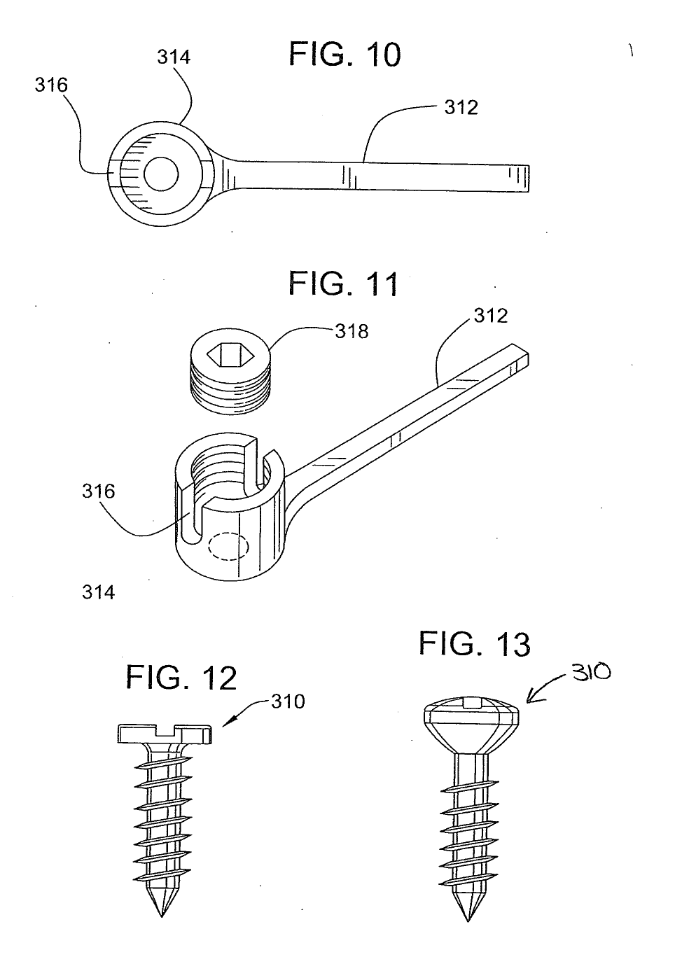

As noted previously, the embodiments shown in FIGS. 4-9 utilize pedicle screws that are substantially equivalent to conventional pedicle screws. A further embodiment of the invention that utilizes a composite head structure 314 that is combined with the rod segment 312 so that a much simpler pedicle screw 310 can be used is shown in FIGS. 10-16. More specifically, in the embodiment of FIGS. 10-16, the head 314 having the slotted recess 316 is incorporated into the rod segments 312. As with the earlier embodiments, this slotted recess 316 is threaded so as to be able to receive a set screw 318. The pedicle screw 310 used in the embodiment of FIGS. 10-16 has a much simpler head that can pass through an opening in the bottom of the head 314 of the rod segment 312 and secure the head to the vertebra.

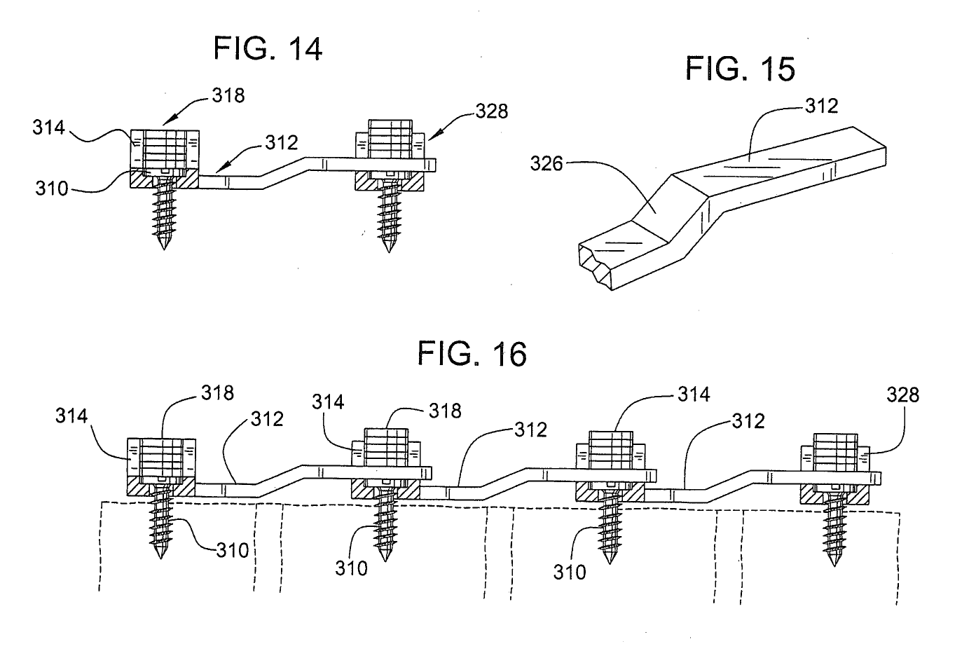

As shown in FIG. 14, fixation is accomplished by placing a conventional pedicle screw 328 at the first level. At the second vertebra, a rod segment 312 is inserted and routed through the slotted recess of the first pedicle screw 328. The head 314 of the rod segment 312 can be secured to the second vertebra by a simple bone screw 310 with a uniquely configured head. In particular, the bone screw 310 can have a generally flat, low profile head such as shown in FIG. 12 that fits into the head 314 of the rod segment 312. In contrast, conventional bone screws have a more cylindrical head similar to a socket head cap screw. A conventional bone screw with a more rounded head, such as shown in FIG. 13 can also be used. If only two vertebrae are to be fixed, then the set screw is placed in the first conventional pedicle screw 328 to clamp and fix the elongated portion of the rod segment 312 within the first pedicle screw. The head 314 of the rod segment 312 is fixed to the bone screw 310 (loosely holding it to the second vertebra) by a similar set screw 318 that drives into the head of the rod segment 312 and clamps on the head of the bone screw 314.

If more vertebrae levels are to be fixed, then more rod segments 312 can be inserted and loosely secured to additional vertebrae (see, e.g., FIG. 16). Each additional rod segment 312 would be installed through a small access port and the elongated section would be routed through the slotted recess 316 in the head 314 of the adjacent rod segment 312. This additional rod segment 312 would then be secured to the vertebra with the bone screw 310. This same procedure can be repeated until all levels requiring fixation have been addressed.

Once all the rod segments 312 have been installed, the spine can then be moved into proper alignment and the set screws 318 tightened. The set screws 318 would compress the elongated segment of an adjacent rod segment 312 against the head of the bone screw 310 and allow for fixation of the vertebrae from one to the next. As adjacent set screws are tightened more levels of the vertebrae would become fixed into a solitary mechanical construction. As with the embodiment in FIGS. 7-9, the rod segments 312 of the FIGS. 10-16 embodiment can have an offset 326 (see FIGS. 15 and 16) in order to compensate for the angular misalignment introduced by the slight offset in height from the head of one rod section 312 to the next. In addition, as described above, the locking of the rod segments 312 can be enhanced by material properties and surface texture and features.

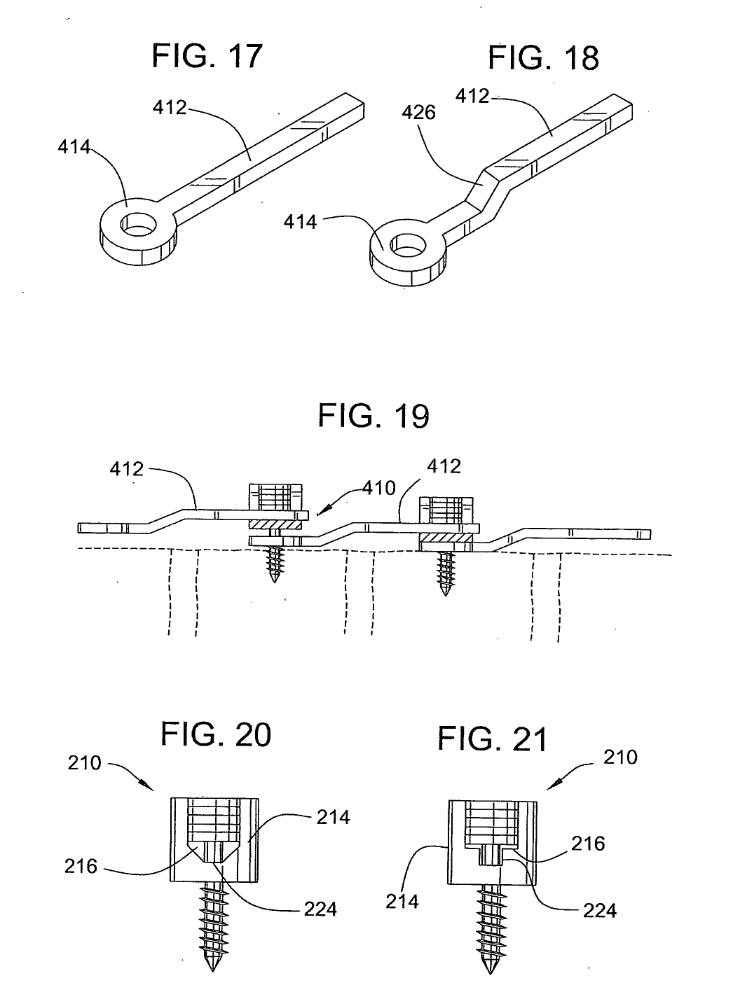

An embodiment somewhat similar to that shown in FIGS. 10-16 is shown in FIGS. 17-19. In the embodiment of FIGS. 17-19, the head 414 of the rod segment 412 has a simple “washer” shape with the elongated portion extending radially away from the edge of the head. This elongated portion can be secured to the bone by a conventional pedicle screw 410. Additional levels can be fixed by applying the rod segments 412 from under the head of one pedicle screw 410 to the slotted recess 416 of the screw at the next vertebra as shown in FIG. 19. As with the above-described embodiments, the rod segment 412 can also have an offset 426 as shown in FIG. 18. Unfortunately, with only the contact pressure of the pedicle screw 410 holding the rod segment 412 in place, it is possible for the fixation to loosen over time. Thus, the embodiment of FIGS. 10-16 is presently preferred because the set screw 318 in each rod segment 312 compresses the adjacent rod segment against the head of the bone screw 310 placed in the vertebra for a more solid and stable construction.

Because the present invention involves transcutaneous assembly of a subcutaneous structure, it is preferred that the assembly be done using articulated tools. Additionally, the assembly procedure requires precise knowledge of where the various components of the system are located at any given time. As a result, assembly of the present invention is particularly well suited to being done by a surgical robot.

In view of the foregoing, a spinal fusion system and method of installation is provided that enables spinal fusion procedures to be performed with minimal invasion of the patient's connective tissue (i.e., no large incisions). By avoiding the removal of large quantities of connective tissue and muscle, the present invention lessens post-operative pain and healing time. Moreover, unlike other so-called minimally spinal fusion systems where only a very limited number of spinal fixation levels can accessed through a single port, the present invention enables fixation of an unlimited number of levels through a single port. In particular, all of the components of the system of the present invention can be inserted under a patient's skin through a single small port and then assembled under the skin.

Transcutaneous of a subcutaneous structure can be accomplished via articulated tools where there is a good knowledge of where the ports are located. Because of this surgical robots are ideal for doing the assembly.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A spinal fusion implant system comprising:
a plurality of links;
a plurality of pedicle screws, each pedicle screw having a head and a depending shank for fixing the pedicle screw to bone, the head having a pair of recesses arranged in side-by-side relation, each recess being configured to receive an end portion of one of the plurality of links, each recess having an associated set screw for clamping the end portion of the respective link in the recess.

2. The spinal fusion implant system according to claim 1 wherein each of the links extends in a substantially straight line.

3. The spinal fusion implant system according to claim 1 wherein each of the links extends in a single plane.

4. The spinal fusion implant system according to claim 1 wherein the recesses in the head of each of the pedicle screws has a slotted configuration.

5. The spinal fusion implant system according to claim 4 wherein the slotted configuration of the recesses in the head of each of the pedicle screws comprises a pair of opposing notches in a sidewall of the respective recess.

6. A method of fixing a plurality of vertebrae together using a plurality of links and a plurality of pedicle screws, each pedicle screw including a head having a pair of recesses with each recess being configured to receive an end portion of one of the plurality of links and each recess having an associated set screw for clamping the end portion of the respective link in the recess, comprising the steps of:
attaching a first pedicle screw to a first vertebrae;
attaching a second pedicle screw to a second vertebrae;
extending a first link between the first and second pedicle screws by inserting a first end portion of the first link in one of the recesses in the head of the first pedicle screw and inserting a second end portion of the first link in one of the recesses in the head of the second pedicle screw;
attaching a third pedicle screw to a third vertebrae;
extending a second link between the second and third pedicle screws by inserting a first end portion of the second link in the other of the recesses in the head of the second pedicle screw and inserting a second end portion of the second link in one of the recesses in the head of the third pedicle screw; and
clamping the first and second ends of the first and second links in position using the set screws associated with the respective recesses in the respective heads of the first, second and third pedicle screws.

7. The vertebrae fixing method of claim 6 wherein the first and second links extend in a substantially straight line.

8. The vertebrae fixing method of claim 6 wherein the first and second links each extend in a single plane.

9. The vertebrae fixing method of claim 6 wherein the first and second pedicle screws are attached such that a first recess of the pair of recesses in the head of each of the first and second pedicle screws are arranged on a first side of an axis connecting the heads of the first and second pedicle screws and a second recess of the pair of recesses in the head of each of the first and second pedicle screws are arranged on the an opposing second side of the axis and wherein the first link extends between the first recesses in the first and second pedicle screws.

10. The vertebrae fixing method of claim 9 wherein the second and third pedicle screws are attached such that the first in the head of the second pedicle screw and a first recess of the pair of recesses in the head of the third pedicle screw are arranged on a first side of an axis connecting the heads of the second and third pedicle screws and the second recess in the head of the second pedicle screw and a second recess of the pair of recesses in the head of the third pedicle screw are arranged on an opposing second side of the axis connecting the second and third pedicle screws and wherein the third link extends between the second recesses in the second and third pedicle screws.

11. A spinal fusion implant system comprising:
a plurality of links, each link having a pair of opposing end portions;
a plurality of pedicle screws, each pedicle screw having a head and a depending shank for fixing the pedicle screw to a bone, the head having a slotted recess configured to receive one end portion of at least two different links, the recess having an associated set screw for clamping the end portions of the respective links in the recess.

12. The spinal fusion implant system according to claim 11 wherein each of the links extends in a substantially straight line.

13. The spinal fusion implant system according to claim 11 wherein each of the links extends in a single plane.

14. The spinal fusion implant system according to claim 11 wherein a first of the pair of end portions of each of the links includes an enlarged head that is receivable in the slotted recesses of the pedicle screws.

15. The spinal fusion implant system according to claim 15 wherein a second of the pair of end portions of each of the links comprises an elongated neck.

16. The spinal fusion implant system according to claim 15 wherein the slotted configuration of the recesses in the head of each of the pedicle screws comprises a pair of opposing notches in a sidewall of the respective recess and the elongated neck of the second end portion of each of the links is insertable through the notches.

17. The spinal fusion implant system according to claim 15 wherein the first and second end portions of the links are substantially flat.

18. The spinal fusion implant system according to claim 15 wherein each of the links includes an offset portion.

19. The spinal fusion implant system according to claim 18 wherein the offset portion has a length corresponding to a thickness of the enlarged head of the respective link.

20. The spinal fusion implant system according to claim 11 wherein the slotted recess of each of the pedicle screws includes a narrower section adjacent a bottom of the recess.

21. The spinal fusion implant system according to claim 20 wherein the narrower section of the slotted recess comprises a sidewall portion that tapers inwardly as the sidewall portion extends towards a bottom of the recess.

22. The spinal fusion implant system according to claim 20 wherein the narrower section of the slotted recess has a V-shaped cross-sectional configuration.

23. The spinal fusion implant system according to claim 20 wherein the narrower section of the slotted recess has a substantially rectangular cross-sectional configuration.

24. A method of fixing a plurality of vertebrae together using a plurality of links and a plurality of pedicle screws, each pedicle screw including a head having a slotted recess configured to receive an end portion of two different links and the recess having an associated set screw for clamping the end portions of the respective links in the recess, comprising the steps of:
attaching a first pedicle screw to a first vertebrae;
attaching a second pedicle screw to a second vertebrae;
extending a first link between the first and second pedicle screws by inserting a first end portion of the first link in the slotted recess in the head of the first pedicle screw and inserting a second end portion of the first link in the slotted recess in the head of the second pedicle screw;
attaching a third pedicle screw to a third vertebrae; and
extending a second link between the second and third pedicle screws by inserting a first end portion of the second link in the slotted recess in the head of the second pedicle screw with the second end portion of the first link and inserting a second end portion of the second link in the slotted recess in the head of the third pedicle screw; and
clamping the first and second ends of the first and second links in position using the set screws associated with the slotted recess in the respective heads of the first, second and third pedicle screws.

25. The spinal fusion implant system according to claim 24 wherein the first end portion of the first link includes an enlarged head that is inserted in the slotted recess of the head of the first pedicle screw.

26. The spinal fusion implant system according to claim 25 wherein the second end portion of the first link includes an elongated neck that is inserted in the slotted recess of the head of the second pedicle screw.

27. The spinal fusion implant system according to claim 26 wherein the first end portion of the second link includes an enlarged head that is inserted in the slotted recess of the head of the second pedicle screw on top of the elongated neck of the second end portion of the first link.

28. The spinal fusion implant system according to claim 27 wherein the slotted recess of the second pedicle screw includes a narrower section adjacent a bottom of the recess into which the elongated neck of the second end portion of the first link is forced by the enlarged head of the first end portion of the second link when the first end portion of the second link is clamped in the recess of the second pedicle screw.

29. A spinal fusion implant system comprising:
a plurality of pedicle screws, each pedicle screw having a screw head and a depending shank for fixing the pedicle screw to a bone;
a plurality of links, each link having a first end portion including an elongated neck and a second end portion including a head having a slotted recess configured to receive the elongated neck of another link, the slotted recess having an opening through which the shank of one of the pedicle screws is insertable and an associated set screw for clamping the elongated neck of the other link in the slotted recess.

30. The spinal fusion system according to claim 29 wherein the screw head of each pedicle screw is substantially flat.

31. The spinal fusion system according to claim 30 wherein the screw head of each pedicle screw is substantially rounded.

31. The spinal fusion system according to claim 30 wherein each link includes an offset portion.

32. A method of fixing a plurality of vertebrae together using a plurality of links and a plurality of pedicle screws, each link including a first end portion comprising an elongated neck and a second end portion comprising a head having a slotted recess configured to receive the elongated neck of another links, the slotted recess having an opening through which a shank of one of the pedicle screws is insertable and an associated set screw for clamping the elongated neck of the other link in the slotted recess, comprising the steps of:
arranging a first link in a desired location relative to a first vertebrae;
attaching the first link to the first vertebrae by inserting a first pedicle screw through the opening in the slotted recess in head of the first link;
arranging a second link in a desired location relative to a second vertebrae;
attaching the second link to the second vertebrae by inserting a second pedicle screw through the opening in the slotted recess in the head of the second link; and
connecting the first link to the second link by inserting the elongated neck of the first link into the slotted recess in the head of the second link and clamping the elongated neck therein with the associated set screw.

33. A spinal fusion implant system comprising:
a plurality of pedicle screws, each pedicle screw having a head and a depending shank for fixing the pedicle screw to a bone; and
a plurality of links, each link including a first end portion including an elongated neck and a second end portion including a head having an opening therethrough through which the shank of one of the pedicle screws is insertable;
wherein the head of each pedicle screw has a slotted recess configured to receive the elongated neck of one link, the recess having an associated set screw for clamping the elongated neck of the respective link in the recess.

34. A method of fixing a plurality of vertebrae together using a plurality of links and a plurality of pedicle screws, each link each link including a first end portion including an elongated neck and a second end portion including a head having an opening therethrough through which a shank of one of the pedicle screws is insertable, each pedicle screw having a head with a slotted recess configured to receive the elongated neck of one link, the recess having an associated set screw for clamping the elongated neck of the respective link in the recess, comprising the steps of:
arranging a first link in a desired location relative to a first vertebrae;
attaching the first link to the first vertebrae by inserting a first pedicle screw through the opening in the head of the first link;
arranging a second link in a desired location relative to a second vertebrae;
attaching the second link to the second vertebrae by inserting a second pedicle screw through the opening in the head of the second link; and
connecting the first link to the second link by inserting the elongated neck of the first link into the slotted recess in the head of the second pedicle screw and clamping the elongated neck therein with the associated set screw.
